(19) United States
(12) Patent Application Publication
(10) Pub. No.: US 2012/0191112 A1
(43) Pub. Date: Jul. 26, 2012

(54) EXPANDABLE CATHETER ASSEMBLY AND CORRESPONDING KIT

(76) Inventor: Paolo Zamboni, Ferrara (IT)

(21) Appl. No.: 13/390,136

(22) PCT Filed: Jul. 27, 2010
(86) PCT No.: PCT/IB2010/053402

§ 371 Date: Apr. 10, 2012

(30) Foreign Application Priority Data
Aug. 12, 2009 (IT) TO2009A000639

(51) Int. Cl. A61B 17/22

(52) U.S. Cl. 606/159

(57) ABSTRACT

A catheter (10) comprises an expandable portion (12) for the treatment of stenotic sites (S) of vessels (JV1, JV2) in the body of a patient. The aforesaid expandable portion (12) includes:—an expandable formation (122), which, when expanded, is able to undergo a localized depression in an area corresponding to an annulus or stenosing ring (S) in the vessel treated (JV1, JV2), detecting the presence of said stenosing ring; and—a cutting formation (124, 126), which can be positioned at the stenosing ring for cutting the stenosing ring so as to bring about yielding of the stenosis produced by said ring. Preferential application is in the treatment of internal jugular veins.

### FIELD OF THE INVENTION

The present disclosure relates to expandable catheters.

The present disclosure has been developed with particular attention paid to the possible use in the treatment of veins, such as for example jugular veins.

### DESCRIPTION OF THE RELATED ART

Expandable catheters are catheters provided with an expandable portion, for example an inflatable balloon, which is set at the distal end of the catheter and can be used for dilating a lumen in the body of a patient.

In the case of a balloon catheter, the catheter in a non-expanded condition, i.e., with the balloon deflated, is positioned in such a way as to bring the balloon to the site to be treated. Then the balloon can be inflated and deflated (possibly repeatedly) to carry out the procedure of expansion and then be finally brought drawn back out of the body of the patient. A procedure that has extensive recourse to catheters of this sort is the procedure known as percutaneous transluminal angioplasty (PTA).

The art concerning expandable catheters is extremely extensive, as is documented by the wide literature available also of a patent nature. In such a context, it is known, for example, to distinguish between catheters of a compliant type and catheters of a non-compliant type.

Documents such as WO-A-2006/124176 or WO-A-2006/062257 in general represent catheters of a compliant type, such as for example balloon catheters, which, if brought into an expanded condition at a stenotic site, tend to assume an hourglass conformation in so far as they are unable to dilate the stenosis completely, which thus impresses a an annular depression or recess, in the balloon, which is otherwise dilated. Documents such as WO-A-2006/086516 or WO-A-2007/075256, instead, describe in general expandable catheters of a non-compliant or high-pressure type. These are for example balloon catheters, which, when expanded at a stenotic site, are in general able to overcome, precisely on account of the high inflation pressure, the resistance opposed by the stenosis.

From documents such as WO-A-2007/136637 or else WO-A-2005/007219 catheters are known provided with an end assembly comprising two balloons in tandem that have in general characteristics one of a compliant type and the other of a non-compliant type.

Recent research in the sector of multiple sclerosis (MS), as documented, for example, in the document No. PCT/IT2008/000129, has demonstrated that a significant percentage of patients affected by multiple sclerosis have malformations of a steno-obstructive type in veins such as the internal jugular veins (IJVs) or the azygos vein.

As regards the jugular veins, the alterations in question, which may give rise to stasis or reflux of blood, primarily present in the form of septa, circular or conical stenoses, or atresiae, typically located at or in the proximity of the jugular-subclavian confluence.

The above stenoses can be treated with traditional angioplasty techniques (PTA) resorting to balloon catheters either of the compliant type or of the non-compliant type.

### PURPOSE AND SUMMARY OF THE INVENTION

The inventor has found that a feasible solution would be recourse to a catheter with an expandable distal portion comprising in tandem a portion of a compliant type and a portion of a non-compliant type that can be expanded independently of one another, for example with inflation pressures in the region of 8 atm (compliant portion) and of 15-20 atm (non-compliant portion).

Added to the above is the possibility of:
i) verifying that the action of dilation exerted by the compliant balloon is sufficient—in the sense that the stenosis yields under the action of the compliant balloon—with the possibility of carrying out the treatment (only) with the balloon of a compliant type; or else
ii) verifying that the action of dilation exerted by the compliant balloon is not sufficient—in the sense that the stenosis does not yield under the action of the compliant balloon—and intervening then with the non-compliant balloon, operating at a higher pressure.

The inventor has, however, been able to verify that, above all in the treatment of stenosis of the internal jugular veins (IJVs) in the course of chronic cerebro-spinal venous insufficiency (CCSVI) that appears to be associated to multiple sclerosis (MS), recourse to traditional angioplasty techniques (albeit performed with recourse—in the context of a single intervention—of a balloon of the high-pressure non-compliant type in the case where the action of a balloon of a compliant type proves insufficient) leads in the follow-up to a significant number of relapses or restenoses.

The object of the present invention is to overcome said drawback by minimizing, and virtually eliminating, the recurrence of said phenomena of restenosis.

According to the present invention, said purpose is achieved thanks to a catheter having the characteristics recalled in the ensuing claims. The invention also regards a kit comprising, together with a catheter of the type specified above, also means of instructions (for example in printed form) for the use of said catheter for the treatment of stenosis of the jugular veins (IJVs).

The claims form an integral part of the technical teaching provided herein in relation to the invention.

Various embodiments are based upon the recognition, by the inventor, of the fact that the cases of restenosis present a predictive signal practically invariably associated to the appearance of a restenosis during follow-up. Said sign is represented by annular stenoses, which, in the course of a treatment of PTA, maintain a fovea (hence a stenotic profile) that causes an impression on a dilating balloon, for example by causing it to maintain—irrespective of the type of balloon and pressure used—an hourglass shape representing an incomplete dilation of an annulus, i.e., of an anomalous circular stricture.

The inventor has encountered said phenomenology also in the presence of dilating pressures of 18-20 atm: there have in fact been encountered stenosing rings of the internal jugular veins (IJVs) that are not dilated even by resorting to such high pressures, and this with an angiographic signal represented by the annular depression or recess fovea that is impressed on the outside of the high-pressure balloon, said depression constituting an angiographic sign that accompanies occurrence of restenosis.

### BRIEF DESCRIPTION OF THE ANNEXED DRAWINGS

The invention will now be described, purely by way of non-limiting example, with reference to the annexed drawings, wherein:

FIG. 1 represents in general the treatment of a jugular vein via catheterism; and

FIGS. 2 to 4 represent schematically the treatment of a stenosis in the jugular veins according to the solution described herein.

### DETAILED DESCRIPTION OF EMBODIMENTS

In the ensuing description, various specific details are illustrated aimed at an in-depth understanding of the embodiments. The embodiments can be made without one or more of the specific details, or with other methods, components, materials, etc. In other cases, known structures, materials, or operations are not illustrated or described in detail so that various aspects of the embodiments will not be obscured.

Reference to “an embodiment” or “one embodiment” in the context of this description is intended to indicate that a particular configuration, structure, or characteristic described in relation to the embodiment is comprised in at least one embodiment. Hence, phrases such as “in an embodiment” or “in one embodiment”, which may be present in different points of this description, do not necessarily refer to one and the same embodiment. Furthermore, particular conformations, structures, or characteristics can be combined adequately in one or more embodiments.

The references used herein are merely for convenience and hence do not define the sphere of protection or the scope of the embodiments.

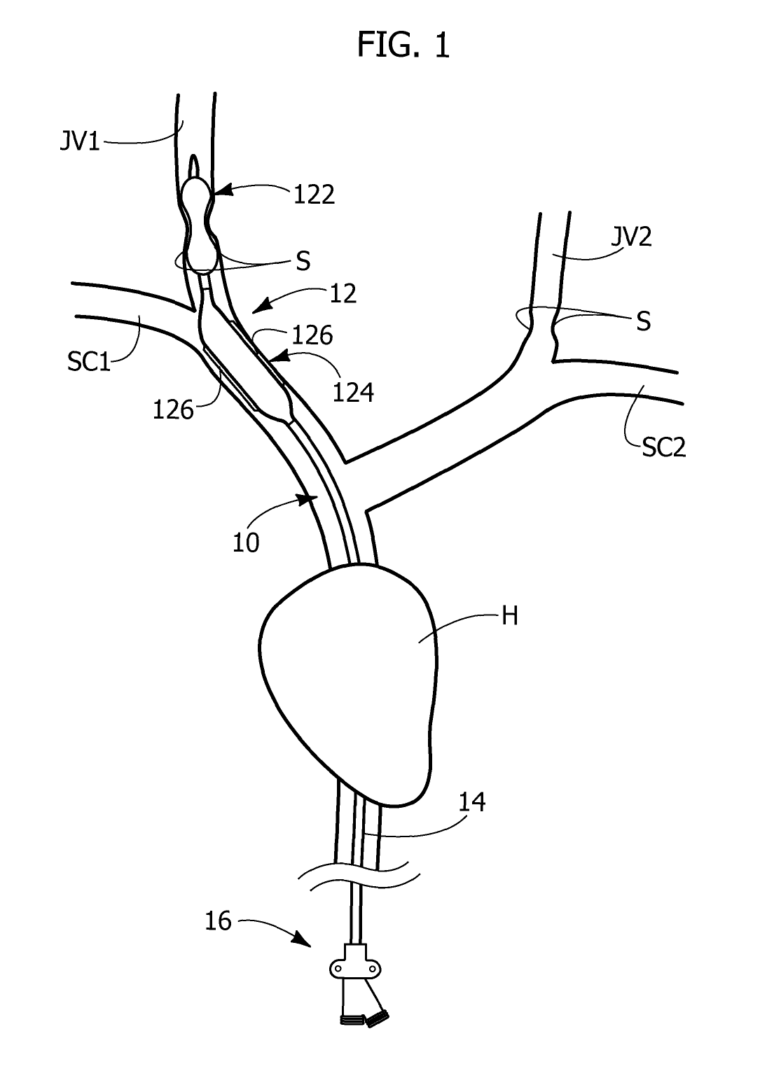

In FIG. 1, the reference number 10 designates as a whole an expandable catheter used for treating (via percutaneous transluminal angioplasty—PTA) localized stenoses in vessels in the body of a patient. In the case considered here, the vessels in question are the jugular veins JV1, JV2 and the stenoses to be treated, designated by S, are localized in the proximity of the confluence of the jugular veins JV1, JV2 in the subclavian veins SC1, SC2.

In the embodiments considered herein, the catheter 10 comprises an expandable portion designated as a whole by 12, situated at the distal end of an elongated flexible structure 14 commonly referred to as shaft of the catheter 10.

In a PTA procedure, the catheter 10 is introduced into the body of the patient, for example via the femoral vein, and is then made to advance along a guide wire in order to locate the expandable portion 12 at the stenotic site S to be treated. Once the expandable portion 12 has reached the site to be treated, it may be expanded by acting on a control device 16 located at the proximal end of the introduction element 14. In various embodiments, the catheter 10 may be introduced via an angiographic guide wire that can enable introduction along paths that are even somewhat tortuous.

In various embodiments, the expandable portion 12 comprises balloon structures that are expandable by inflation. The expansion means 16 are in this case in the form of pumping means which are able to convey along the introduction structure 14, via ducts made in the structure 14 itself, a pressurized fluid (for example, physiological solution) that is able to inflate the expandable portion 12.

A PTA procedure can comprise one or more cycles of inflation/deflation of the expandable part 12. Once the procedure has been completed, the expandable portion 12 can finally be inflated so as to enable recall of the catheter 10 outside the body of the patient, causing it to slide backwards along the guide wire, which is finally also extracted from the body of the patient.

In these general terms, the PTA procedure and the characteristics of the catheter 10 just described are to be deemed known to the art and such as not to require a detailed description herein. Except where otherwise indicated in what follows, what has been said above applies also to the technology regarding the production of the catheter 10 and of the parts that make it up (for example, the inflatable balloons).

In this regard, persons skilled in the sector will understand that, whilst the embodiments considered herein refer to expandable structures in the form of a balloon, the scope of the present disclosure is such as to embrace expandable structures of a different nature, such as, for instance, mechanically expandable structures, for example (micro)tubes with slitted walls which can expand radially in the presence of an axial contraction induced via a flexible tensile element extending along the introduction structure 14 and/or self-expandable structures comprising shape-memory materials or superelastic materials, such as the material known as Nitinol, which is well known for applications in the field of devices for angioplasty, such as stents.

In the embodiments considered herein, the expandable portion 12 of the catheter 10 comprises two expandable formations 122, 124 set in tandem.

In various embodiments, the arrangement in tandem comprises two expandable formations 122, 124 (for simplicity, in what follows constant reference will be made to two balloons, without prejudice to what has been said above as regards other possible embodiments) spaced apart from one another and set, respectively, in a distal position and in a proximal position, so that the two formations 122, 124 are each expandable independently of the other.

The terms “distal” and “proximal” refer to the direction of introduction of the catheter 10. The embodiments considered herein envisage that the catheter 10 is introduced into the body of the patient via the femoral vein and then made to advance towards and through the heart H in order to locate the expandable portion 12 in a position corresponding to one or the other of the jugular veins JV1, JV2.

In various embodiments, the balloons 122, 124 (it should be recalled once again that one or both of said expandable formations can be made with structures different from an expandable balloon) have a length—measured in the longitudinal direction with respect to the catheter 10—in the region of 3-4 cm. In the case of the balloon 124 also shorter lengths may be hypothesized, for example 1.3 cm.

In various embodiments, both of the expandable formations 122, 124, when expanded, have a diameter of between 8 and 12 mm.

In various embodiments, the catheter 10 has a “useful” length (i.e., the length that can be used for catheterism) of approximately 120 cm.

In various embodiments, the distal formation 122 is of the non-compliant type, for example, a high-pressure balloon (HPB), whilst the proximal formation 124 is an inflatable balloon of a compliant type. The meaning of “non-complaint” and “compliant” has been discussed in the introductory part of the present description.

In various embodiments, the proximal balloon of a compliant type 124 has a (maximum) inflation pressure of approximately 8 atm. In various embodiments, the distal balloon of the non-compliant type has a (maximum) inflation pressure of 15-20 atm.

Once again, whereas the embodiments considered herein envisage that the balloon 122 is situated in a distal position as compared to the balloon 124, various embodiments can envisage that the balloon 122 is located in a proximal position as compared to the balloon 124.

In various embodiments, the formation 124 (which, as will once again be recalled, does not necessarily assume the characteristics of a balloon and in itself may even not be of an expandable type) is a structure of a cutting type constituted, for example, by a so-called cutting balloon (CB).

Cutting structures of this type are extensively known in the art for carrying out treatments of various nature, as documented for example in EP-A-0 147 192, EP-A-0 288 833, EP-A-0 289 319, EP-A-0 355 200, EP-A-0 419 154, EP-A-0 619 986, EP-A-1 610 841, EP-A-1 628 703, EP-A-1 734 875, EP-A-1 809 361, EP-A-1 314 467, EP-A-1 830 915, EP-A-1 850 765, and EP-A-1 962 696. In various embodiments, the formation 124 can comprise a balloon resembling the product available under the commercial name of Peripheral Cutting Balloon™ manufactured by the company Boston Scientific Scimed, Inc. and designed to be used as atherotome, i.e., as endovascular cutting device for arteriosclerotic plaques. Said balloon is provided with cutting blades with longitudinal development along the margin of the balloon.

In the case of the application considered herein (cutting/incision of an annulus in a vein, as will emerge more clearly in what follows) an overall length of the balloon 124 of 1.3 cm, with a corresponding reduction of the length of the cutting blades may be hypothesized. In various embodiments, the cutting blades, designated by 126 in the drawings, are two in number, mounted in diametrally opposite positions (i.e., at 180° apart from one another) on an ovoid frame that at rest is fixed with respect to the deflated balloon. When the balloon 124 is dilated, the elastic material expands until it enables contact of the cutting blades 126 with the wall of the vessel to be treated, whilst when the balloon 124 is deflated the blades 126 return into the resting position. As will be described more fully in what follows, with a subsequent rotation of the catheter through 90° the blades 126 can carry out a second cut.

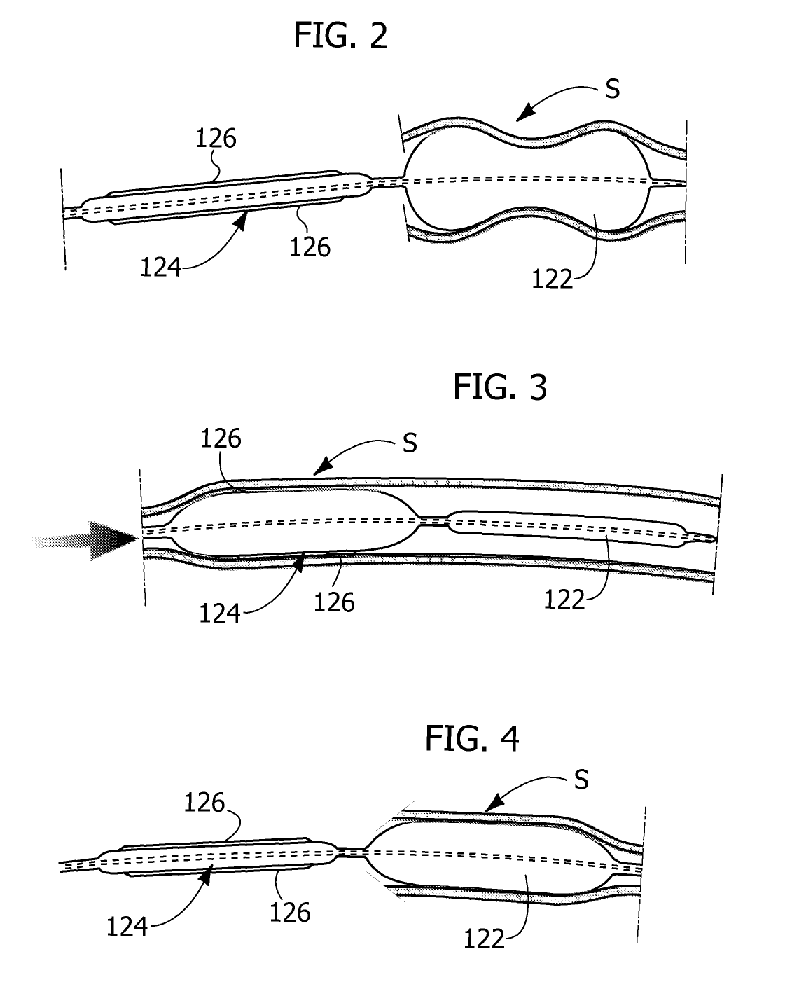

FIGS. 2 to 4 illustrate schematically execution of a procedure of PTA that may be implemented with the embodiments considered herein.

As first step, the catheter 10 is introduced so as to bring the distal balloon 122 to the stenotic site S to be treated.

A first procedure of PTA is then performed by expanding the balloon 122 as illustrated schematically both in FIG. 1 and in FIG. 2.

The treatment may be carried out (in the embodiments considered herein the balloon 122 is a balloon of the non-compliant type, hence a high-pressure balloon HPB) bringing the inflation pressure up to values of between 15-20 atm. Said values of pressure (and in particular values in the upper part of said range, such as for example 18-20 atm) can be reached by coupling to the inflation device 16 situated at the proximal end of the catheter an inflation device such as a pistol-shaped syringe or else a motor-driven syringe that can be actuated by the operator without making any effort.

In the case where the corresponding angiographic signal shows that during execution of this first PTA procedure (it will be recalled that said procedure usually comprises a cycle of operations of inflation and deflation, for example of a duration of 30-60 seconds repeated in an alternating way) the balloon 122 expands completely and is hence not “impressed” by the stenosis S (in other words, the balloon 122, when expanded, does not present an hourglass structure or in general a portion restricted by the stenosis S) the treatment can also be considered completed with this single step.

If instead the angiographic signal shows the persistence of an annular depression or recess impressed on the outside of the balloon 122 even at the highest values of inflation pressure (as illustrated schematically in FIG. 1 and FIG. 2), there exist reasons to think that the treatment leaves open the risk of restenosis during follow-up. In said conditions, the balloon 122 consequently functions, so to speak, as probe that highlights exposure to said risk, likewise detecting, in addition to the presence of the stenosing ring that causes the depression in the balloon itself, also the position thereof.

In this case, as schematically represented in the sequence of FIGS. 2 and 3, by acting on the catheter it is possible to cause the balloon 124 provided with the cutting blades 126 to advance towards the stenosis S, precisely where the stenosing ring is present, the position of which has been detected by the balloon 122 functioning as probe.

The balloon 124 is then dilated, as illustrated in FIG. 3, so that, by incising the wall of the vessel, the blades 126 cut the fibres of the stenosing ring S.

In the case of ???two diametrally opposite blades 126, the annulus is cut in two diametrally opposite positions. As already anticipated, the operation of dilation of the balloon 124 can be repeated with the possibility, on the part of the operator, of rotating the catheter—and hence the balloon 124 with the blades 126—between one dilation and the next, for example causing it to rotate through 90° in a clockwise or counterclockwise direction, so that two successive actions of dilation bring the cutting blades 126 to act on the stenosing ring in four points angularly spaced apart by 90°.

The operator can act until he detects detects visually yielding of the stenosis S, which is no longer capable of impressing the depression on the balloon 124.

In various embodiments, the use as expandable structure 124 carrying the cutting blades 126, of a balloon of a compliant type, with a low inflation pressure, renders practically certain the fact that, at the moment when the stenosis S no longer resists the action of dilation of a low-pressure balloon, much less will it resist the action of a high-pressure non-compliant balloon.

In this way, as schematically represented in FIG. 4, the operator can bring the distal balloon 122 (HPB) back into a position corresponding to the stenosis by carrying out a traditional angioplasty—after cutting of the stenosing ring—in order to remodel the vessel, thus concluding the intervention of PTA.

In various embodiments, the cutting blades 126 of the expandable structure 124 can be made in such a way that they can be controlled selectively, not only in the working condition described previously, where the cutting blades 126 project from the expandable structure 124 and are able to perform the cutting action, but also in a retracted condition, where the blades 126 are not able to act as cutting elements.

In the case where the expandable formation 124 presents said characteristics, once the action of cutting of the stenosing ring has been performed, the blades 126 can be brought into the retracted condition so that the expandable structure 124 can possibly be used for remodelling the vessel at the end of the treatment, without bringing the expandable structure 122 back into the position of the stenosis.

Of course, without prejudice to the principle of the invention, the details of construction and the embodiments may vary, even significantly, with respect to what has been illustrated herein purely by way of non-limiting example, without thereby departing from the scope of the invention, as defined by the annexed claims.

1. A catheter including an expandable portion for treating stenotic sites (S) in vessels in a patient's body, wherein said expandable portion includes:
an expandable formation which, when expanded, is able to exhibit a fovea at an annulus (S) stenosing a vessel treated, thus detecting the presence of said stenosing annulus, and
a cutting formation locatable at said stenosing annulus to cut said stenosing annulus to produce yielding of the stenosis caused by said annulus.

2. The catheter of claim 1, wherein said expandable formation is an expandable balloon.

3. The catheter of claim 2, wherein said expandable formation is a high pressure expandable balloon, preferably having an inflating pressure between 15 and 20 atmospheres.

4. The catheter of claim 1, wherein said cutting formation is an expandable formation.

5. The catheter of claim 4, wherein said cutting formation includes an expandable balloon.

6. The catheter of claim 5, wherein said cutting formation includes a low pressure expandable balloon, preferably having an inflating pressure up to 6 atmospheres.

7. The catheter of claim 1, wherein said cutting formation includes at least one pair of diametrically opposed cutting elements.

8. The catheter of claim 1, wherein said cutting formation includes cutting elements actuatable to a recessed condition, wherein said cutting elements are inhibited from said cutting action.

9. The catheter of claim 1, wherein said expandable formation is located distally of said cutting formation.

10. A kit including:
the catheter of claim 1, and
instruction means instructing the use of said catheter for treating stenosis in the jugular veins.
